(19) United States
(12) Patent Application Publication
(10) Pub. No.: US 2021/0346637 A1
(43) Pub. Date: Nov. 11, 2021

(54) Pediatric Nebulizer Assembly

(71) Applicant: Christopher Hearn, Pasadena, CA (US)

(72) Inventor: Christopher Hearn, Pasadena, CA (US)

(21) Appl. No.: 16/870,268

(22) Filed: May 8, 2020

(51) Int. Cl. A61M 16/14; A61M 16/04; A61M 16/10; A61M 16/08; A61M 16/00

(52) U.S. Cl. CPC A61M 16/14; A61M 16/0488; A61M 16/1005; A61M 2202/0208; A61M 16/0012; A61M 2205/123; A61M 2206/16; A61M 16/0833

(57) ABSTRACT

A pediatric nebulizer assembly includes an oxygen hose and a nebulizer containing a fluid medication. The nebulizer engages the oxygen hose to nebulize the fluid medication into oxygen for delivering outwardly through the outlet. A tee fitting releasably engages the nebulizer to receive the nebulized fluid medication and the oxygen. A mouthpiece is attachable to the tee fitting to receive the nebulized fluid medication and the oxygen for inhalation by a patient. A rifled barrel is attachable to the elbow fitting when the mouthpiece is not attached to the tee fitting to receive the nebulized fluid medication and the oxygen. In this way the rifled barrel enhances delivery to a pediatric patient that may be not be capable of inhaling through the mouthpiece.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to nebulizer devices and more particularly pertains to a new nebulizer device for nebulizing an infant or toddler.

### (2) Description of Related Art Including Information Disclosed Under 37 CFR 1.97 and 1.98

The prior art relates to nebulizer devices including a package system that has a plurality of tearable panels for easy access to contents of the package system. The prior art discloses a bellows for manually ventilating a patient. The prior art also discloses an albuterol nebulizer that includes a nebulizer and a mask for wearing on a patient. The prior art discloses a nebulizer kit that includes a nebulizer, a pre-measured amount of a liquid medication and a mask for inhaling the nebulized fluid medication. The prior art discloses a portable nebulizer that employs ambient air to nebulize a fluid medication for inhalation.

### BRIEF SUMMARY OF THE INVENTION

An embodiment of the disclosure meets the needs presented above by generally comprising an oxygen hose and a nebulizer containing a fluid medication. The nebulizer engages the oxygen hose to nebulize the fluid medication into oxygen for delivering outwardly through the outlet. A tee fitting releasably engages the nebulizer to receive the nebulized fluid medication and the oxygen. A mouthpiece is attachable to the tee fitting to receive the nebulized fluid medication and the oxygen for inhalation by a patient. A rifled barrel is attachable to the elbow fitting when the mouthpiece is not attached to the tee fitting to receive the nebulized fluid medication and the oxygen. In this way the rifled barrel enhances delivery to a pediatric patient that may not be capable of inhaling through the mouthpiece.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a kit view of a pediatric nebulizer assembly according to an embodiment of the disclosure.

FIG. 2 is a cross sectional view taken along line 2-2 of FIG. 1 of an embodiment of the disclosure.

FIG. 3 is perspective view of a storage bag of an embodiment of the disclosure.

### DETAILED DESCRIPTION OF THE INVENTION

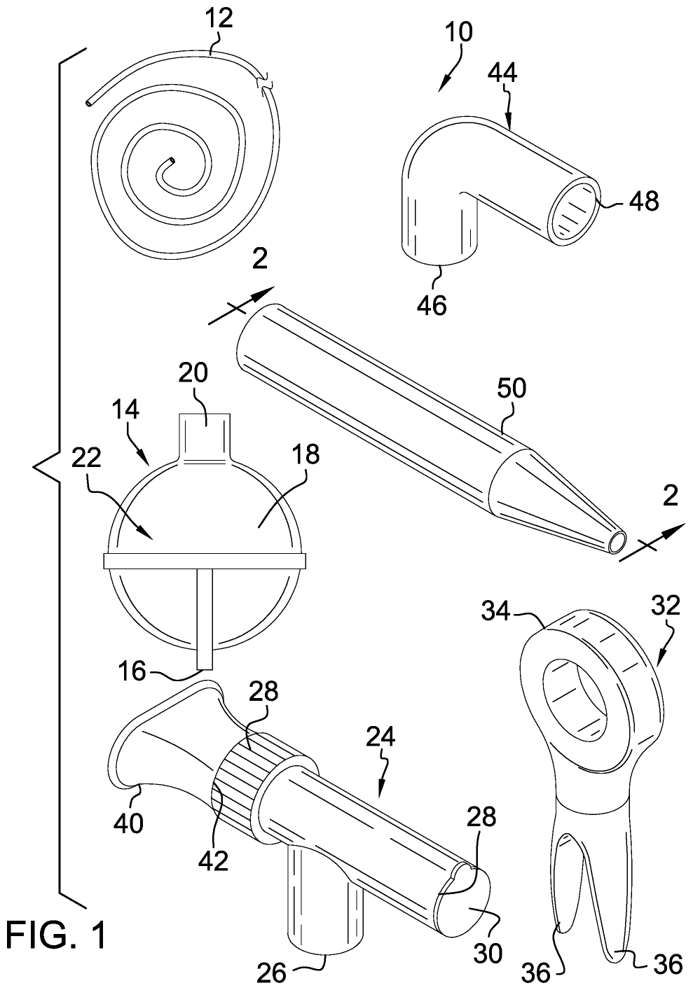

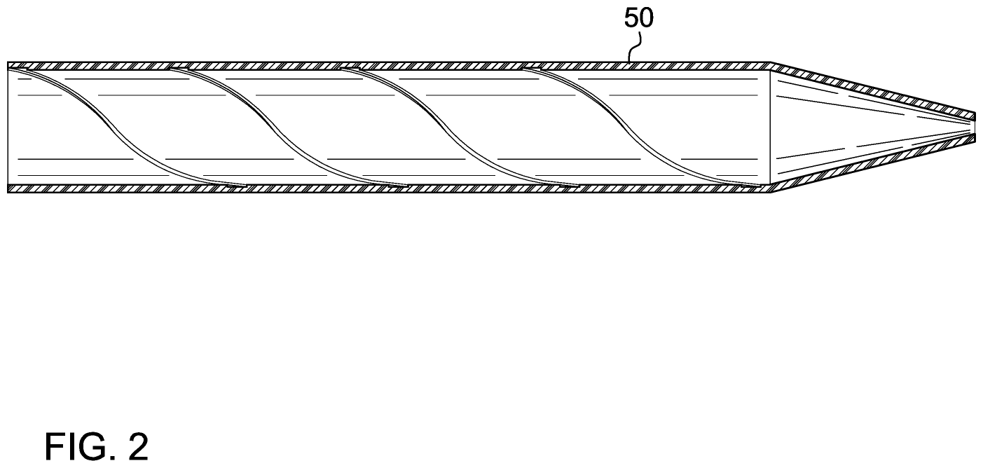

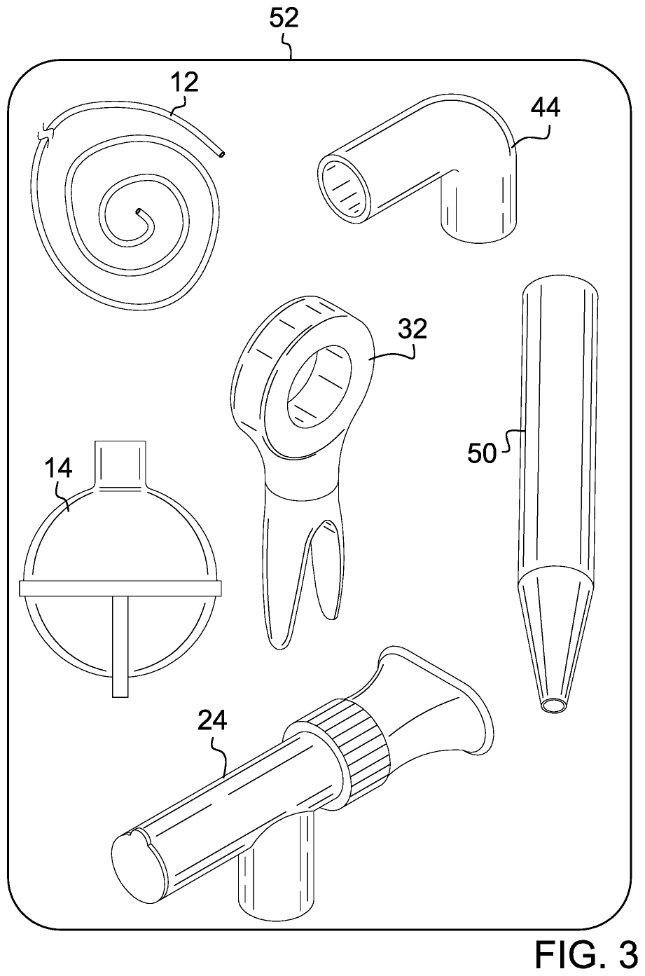

With reference now to the drawings, and in particular to FIGS. 1 through 3 thereof, a new nebulizer device embodying the principles and concepts of an embodiment of the disclosure and generally designated by the reference numeral 10 will be described.

As best illustrated in FIGS. 1 through 3, the pediatric nebulizer assembly 10 generally comprises an oxygen hose 12 that is comprised of an oxygen safe material thereby facilitating the oxygen hose 12 to be fluidly coupled to an oxygen supply. The oxygen supply may be an oxygen tank in an ambulance or other medical environment. A nebulizer 14 is included that has an inlet 16, a reservoir 18 that is in fluid communication with the inlet 16 and an outlet 20 that is in fluid communication with the reservoir 18. The reservoir 18 contains a fluid medication 22, such as albuterol, or other type of medication that is commonly inhaled. The inlet 16 engages the oxygen hose 12 to nebulize the fluid medication 22 into the oxygen for delivering outwardly through the outlet 20. The nebulizer 14 may be a pomegranate type of nebulizer or the like.

A tee fitting 24 is included that has an input 26 and a pair of outputs 28 that is each in fluid communication with the input 26. The input 26 engages the outlet 20 of the nebulizer 14 to receive the nebulized fluid medication 22 and the oxygen. Each of the outputs 28 is oriented to extend along a line that is oriented perpendicular to a line extending through the input 26. Moreover, the outputs 28 are positioned on opposite ends of the tee fitting 24 from each other. A cap 30 is removably attachable to a respective one of the outputs 28 on the tee fitting 24 for closing the respective output 28. In this way the cap 30 inhibits the nebulized fluid medication 22 and the oxygen from passing through the respective output 28.

A pinch mount 32 is provided and the pinch mount 32 includes a ring 34 that is pivotally coupled to a pair of pinchers 36. Each of the pinchers 36 is biased toward each other to grip a support for retaining the pinch mount 32 on the support. The tee fitting 24 is extendable through the ring 34 thereby facilitating the nebulizer 14 and the tee fitting 24 to be mounted to the support. The support may be a child car seat, a bassinette or any other carrier for an infant or toddler.

A mouthpiece 40 is included that is attachable to the tee fitting 24 to receive the nebulized fluid medication 22 and the oxygen for inhalation by a patient. The mouthpiece 40 has an input 42 that engages a respective one of the outputs 28 on the tee fitting 24. Additionally, the mouthpiece 40 may be a nebulizer mouthpiece of any conventional design. An elbow fitting 44 is attachable to the tee fitting 24 when the mouthpiece 40 is not coupled to the tee fitting 24 to receive the nebulized fluid medication 22 and the oxygen. The elbow fitting 44 has an inlet 46 that is oriented perpendicular to an outlet 48. The inlet 46 of the elbow fitting 44 engages a respective one of the outputs 28 on the tee fitting 24.

A rifled barrel 50 is provided and the rifled barrel 50 is attachable to the elbow fitting 44 when the mouthpiece 40 is not attached to the tee fitting 24 receive the nebulized fluid medication 22 and the oxygen. The rifled barrel 50 tapers to a blunt point to increase pressure of the nebulized fluid medication 22 and the oxygen with the Venturi effect. Moreover, the rifled barrel 50 is internally rifled to induce rotation into the flow of the nebulized fluid medication 22 and the oxygen. In this way the rifled barrel 50 enhances delivery to a pediatric patient that may be not be capable of sucking on the mouthpiece 40. Thus, an infant can be nebulized to treat a breathing emergency.

A storage bag 52 is provided and the oxygen hose 12, the nebulizer 14, the tee fitting 24, the cap 30, the pinch mount 32, the mouthpiece 40, the elbow fitting 44 and the rifled barrel 50 are stored in the storage bag 52. Additionally, the storage bag 52 is colored and structured to match existing oxygen safe storage bags. A ventilator can be coupled to the tee fitting 24 to manually ventilate the patient if the patient is not capable of inhaling on their own. In this way the nebulized fluid medication 22 and the oxygen can be delivered to a patient that is in danger of suffocating due to severe allergic reactions or other biological processes that impede breathing.

In use, oxygen hose 12 is coupled to the nebulizer 14 and the nebulizer 14 is coupled to the tee fitting 24. The mouthpiece 40 is coupled to the tee fitting 24 when the patient is old enough to inhale through the mouthpiece 40 without assistance. The rifled barrel 50 is coupled to the tee fitting 24 when the patient is an infant or toddler that cannot inhale through the mouthpiece 40 without assistance. In this way the rifled barrel 50 facilitates an infant to be more effectively nebulized in a breathing emergency as compared to existing nebulizers that are designed for older children and adults. The pinch mount 32 can be attached to the support thereby facilitating the tee fitting 24 and the nebulizer 14 to be mounted to the support. In this way a caregiver does not have to use both hands to administer the nebulized fluid medication 22 and the oxygen.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A pediatric nebulizer assembly for delivering oxygen and a nebulized medication to a pediatric patient, said assembly comprising:
an oxygen hose being comprised of an oxygen safe material thereby facilitating said oxygen hose to be fluidly coupled to an oxygen supply;
a nebulizer containing a fluid medication, said nebulizer engaging said oxygen hose wherein said nebulizer is configured to nebulize the fluid medication into the oxygen for delivering outwardly through said outlet;
a tee fitting releasably engaging said nebulizer wherein said tee fitting is configured to receive the nebulized fluid medication and the oxygen;
a pinch being configured to grip a support for retaining said pinch mount on the support, said ring engaging said tee fitting thereby facilitating said nebulizer and said tee fitting to be mounted to the support;
a mouthpiece being attachable to said tee fitting wherein said mouthpiece is configured to receive the nebulized fluid medication and the oxygen for inhalation by a patient;
an elbow fitting being attachable to said tee fitting when said mouthpiece is not coupled to said tee fitting wherein said elbow fitting is configured to receive the nebulized fluid medication and the oxygen; and
a rifled barrel being attachable to said elbow fitting when said mouthpiece is not attached to said tee fitting wherein said rifled barrel is configured to receive the nebulized fluid medication and the oxygen thereby enhancing delivery to a pediatric patient that may be not be capable of inhaling through said mouthpiece.

2. The assembly according to claim 1, wherein said nebulizer has an inlet, a reservoir being in fluid communication with said inlet and an outlet being in fluid communication with said reservoir, said reservoir containing the fluid medication, said inlet engaging said oxygen hose wherein said nebulizer is configured to nebulize the fluid medication into the oxygen for delivering outwardly through said outlet.

3. The assembly according to claim 2, wherein said tee fitting has an input and a pair of outputs each being in fluid communication with said input, said input engaging said outlet of said nebulizer wherein said tee fitting is configured to receive the nebulized fluid medication and the oxygen, each of said outputs being oriented to extend along a line being oriented perpendicular to a line extending through said input, said outputs being positioned on opposite ends of said tee fitting from each other.

4. The assembly according to claim 3, further comprising a cap being removably attachable to a respective one of said outputs on said tee fitting for closing said respective output wherein said cap is configured to inhibit the nebulized fluid medication and the oxygen from passing through said respective output.

5. The assembly according to claim 1, wherein said pinch mount has a ring being pivotally coupled to a pair of pinchers, each of said pinchers being biased toward each other wherein each of said pinchers is configured to grip a support for retaining said pinch mount on the support, said ring having said tee fitting being extendable therethrough thereby facilitating said nebulizer and said tee fitting to be mounted to the support.

6. The assembly according to claim 3, wherein said elbow fitting has an inlet being oriented perpendicular to an outlet, said inlet of said elbow fitting engaging a respective one of said outputs on said tee fitting.

7. The assembly according to claim 1, wherein said rifled barrel tapers to a blunt point wherein said rifled barrel is configured to increase pressure of the nebulized fluid medication and the oxygen with the Venturi effect.

8. The assembly according to claim 1, wherein said rifled barrel is internally rifled wherein said rifled barrel is configured to induce rotation into the flow of the nebulized fluid medication and the oxygen.

9. A pediatric nebulizer assembly for delivering oxygen and a nebulized medication to a pediatric patient, said assembly comprising:
an oxygen hose being comprised of an oxygen safe material thereby facilitating said oxygen hose to be fluidly coupled to an oxygen supply;
a nebulizer having an inlet, a reservoir being in fluid communication with said inlet and an outlet being in fluid communication with said reservoir, said reservoir containing a fluid medication, said inlet engaging said oxygen hose wherein said nebulizer is configured to nebulize the fluid medication into the oxygen for delivering outwardly through said outlet;
a tee fitting having an input and a pair of outputs each being in fluid communication with said input, said input engaging said outlet of said nebulizer wherein said tee fitting is configured to receive the nebulized fluid medication and the oxygen, each of said outputs being oriented to extend along a line being oriented perpendicular to a line extending through said input, said outputs being positioned on opposite ends of said tee fitting from each other;
a cap being removably attachable to a respective one of said outputs on said tee fitting for closing said respective output wherein said cap is configured to inhibit the nebulized fluid medication and the oxygen from passing through said respective output;
a pinch mount having a ring being pivotally coupled to a pair of pinchers, each of said pinchers being biased toward each other wherein each of said pinchers is configured to grip a support for retaining said pinch mount on the support, said ring having said tee fitting being extendable therethrough thereby facilitating said nebulizer and said tee fitting to be mounted to the support;
a mouthpiece being attachable to said tee fitting wherein said mouthpiece is configured to receive the nebulized fluid medication and the oxygen for inhalation by a patient, said mouthpiece having an input that engages a respective one of said outputs on said tee fitting;
an elbow fitting being attachable to said tee fitting when said mouthpiece is not coupled to said tee fitting wherein said elbow fitting is configured to receive the nebulized fluid medication and the oxygen, said elbow fitting having an inlet being oriented perpendicular to an outlet, said inlet of said elbow fitting engaging a respective one of said outputs on said tee fitting; and
a rifled barrel being attachable to said elbow fitting when said mouthpiece is not attached to said tee fitting wherein said rifled barrel is configured to receive the nebulized fluid medication and the oxygen, said rifled barrel tapering to a blunt point wherein said rifled barrel is configured to increase pressure of the nebulized fluid medication and the oxygen with the Venturi effect, said rifled barrel being internally rifled wherein said rifled barrel is configured to induce rotation into the flow of the nebulized fluid medication and the oxygen thereby enhancing delivery to a pediatric patient that may be not be capable of inhaling on said mouthpiece.
